(19) United States
(12) Patent Application Publication
(10) Pub. No.: US 2003/0104614 A1
(43) Pub. Date: Jun. 5, 2003

(54) Micropatterning surfaces of polymeric substrates

(76) Inventors: Kathryn E. Uhrich, Plaintield, NJ (US); Helen M. Buettner, West Windsor, NJ (US); Kristine Schmalenberg, Dunellen, NJ (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/215,435

(22) Filed: Aug. 9, 2002

Related U.S. Application Data
(63) Continuation of application No. PCT/US01/04842, filed on Feb. 12, 2001.
(60) Provisional application No. 60/181,763, filed on Feb. 11, 2000.

(51) Int. Cl. C12N 5/02; C12M 1/00

(52) U.S. Cl. 435/325; 435/289.1

(57) ABSTRACT

The present invention is directed to an article which has a pattern of biologically active molecules stably adsorbed directly onto a polymeric substrate. The present invention also provides methods for preparing a pattern of biologically active molecules on the surface of a polymeric substrate, which include exposing a polymeric substrate to conditions that increase the polarity of a surface of the polymeric substrate, and contacting that surface with a stamp that includes a micron-sized pattern coated with biologically active molecules. The present invention also provides a method to spatially modulate the growth of a cell which includes contacting a cell with an article of the present invention for a time and under conditions sufficient to adhere the cell to the biologically active molecules and to grow the cell along the micron-sized pattern of biologically active molecules on the polymeric substrate.

### CROSS-REFERENCE TO RELATED APPLICATION(S)

This application is a continuation under 35 U.S.C. 111(a) of International Application No. PCT/US01/04842 filed Feb. 12, 2001, which claims priority from U.S. Provisional Patent Application No. 60/181,763, filed Feb. 11, 2000, which applications are incorporated herein by reference.

### FIELD OF THE INVENTION

The present invention relates generally to the field of making patterns of biologically active molecules on polymeric substrates. More specifically, it relates to an article with a pattern of biologically active molecules on the surface of a polymeric substrate. Methods of making and using such articles are also provided by the present invention. According to the present invention, micropatterned biomolecules have a number of applications, for example, modulating cell-substrate interactions, spatially directing cell growth, tissue regeneration, combinatorial screening strategies, and multiple analytical biosensors. Some of the most exciting applications of patterned biologically active molecules on biocompatible substrates include modulating the growth of cells through cell-substrate interactions. According to the present invention, cell growth can be modulated spatially both in vivo and in vitro, so that for example, an injured tissue can be regenerated into an appropriate, functional shape, and a severed nerve cell connection can be repaired by encouraging nerve cells to grow across a gap in the connection. Also according to the present invention, the rate of cell growth can be modulated through use of biologically active molecules that increase cell growth, e.g. growth factors. Hence, the present invention is directed to these types of methods for using the inventive articles provided herein.

### BACKGROUND OF THE INVENTION

Micropatterning is a technique that has been in use for decades in the computer industry for patterning of microchips. More recently, patterning of substrates has been contemplated for biological applications including biological assays, medical implants and articles for adhering and growing cells. Most of such micropatterning techniques have involved placing patterns on inorganic, non-polymeric substrates such as glass.

High-resolution photolithography, sometimes referred to as microlithography, and focused laser methods have been described for patterning surfaces with molecular layers (Dontha et al., Anal. Chem., 69:2619 (1997)); Kleinfeld et al., J. Neurosci., 8:4098 (1988)). However, photolithography requires the use of harsh solvents and bases which are incompatible with many biological molecules, and the laser method uses an interference technique that does not permit generation of patterns of arbitrary complexity (James et al., Langmuir, 14:741 (1998)).

Microcontact printing is another relatively new technique that has been used to produce micron-sized features on inorganic surfaces. Using this technique, inorganic substrates, such as glass and silicon oxide, have been micropatterned by stamping inks such as alkanethiols on their surface (St. John et al., Anal. Chem., 70:1108 (1998); and James et al., Langmuir, 12:741 (1998)). In this method, a high resolution protein pattern is applied to the surface using a stamp made from poly(dimethylsiloxane) secured to a glass backing material.

One instance has been described where a biocompatible polymer having a micro-patterned surface was produced (WO 99/36107). In this case, a ligand was attached to a biocompatible polymer though a specific biotin-avidin linkage.

Methods for direct and stable adsorption of biologically active molecules onto the surface of a polymeric substrate have not yet been reported. Such methods would minimize the number of constituents in the article, thereby avoiding adverse side effects, non-specific binding and cross-reactivity as well as simplifying and reducing the costs of manufacturing. Accordingly, a need exists for an article having a biologically active molecule directly adsorbed onto a polymeric substrate and for a method to produce such an article.

### SUMMARY OF THE INVENTION

The present invention provides an article which has a pattern of biologically active molecules stably adsorbed on a polymeric substrate.

The present invention also provides methods for preparing a pattern of biologically active molecules on the surface of a polymeric substrate, which include exposing a polymeric substrate to conditions that increase the polarity of a surface of the polymeric substrate, and contacting that surface with a pre-selected pattern of biologically active molecules.

The present invention further provides a method to spatially modulate the growth of a cell which includes contacting a cell with an article of the present invention for a time and under conditions sufficient to adhere the cell to the biologically active molecules and to grow the cell along the micron-sized pattern of biologically active molecules on the polymeric substrate. Cells contemplated for use in the present methods nerve cells, epithelial cells, mesenchymal stem cells, fibroblast cells, and other cell types. The present methods can also include a step of implanting the article a mammal or human.

The present invention still further provides a method to regenerate a tissue which includes contacting cells of the tissue to be regenerates with an article of the invention for a time and under conditions sufficient to adhere the cells to biologically active molecules stably adsorbed to a polymeric substrate surface of the article and to grow the cells in a pre-selected pattern of biologically active molecules on the polymeric substrate. Examples of a pre-selected pattern useful for regenerating tissues include a shape missing from the tissue and a normal shape for the tissue.

The present invention also provides a method for patterning a surface of a polymeric substrate with micron-sized features which includes stamping a surface of a polymeric substrate with a natural or synthetic biologically active molecule.

The present invention further provides polymeric substrates having a surface patterned with micron-sized features which is prepared by stamping the surface of the polymeric substrate with a natural or synthetic polymer-based ink or a biologically active molecule.

The present invention still further provides a device for guided tissue regeneration which comprises a biodegradable or nondegradable polymeric substrate having a surface patterned with micron-sized features which is prepared by stamping the surface of the biodegradable or nondegradable polymeric substrate with a natural or synthetic polymer-based ink.

Preferred embodiments of the present invention include methods for patterning polymeric substrates with micron-sized features which involve:

(a) preparing a stamp with the selected micron-sized pattern;

(b) exposing the stamp and a polymeric substrate to be patterned to conditions which increase their polarity;

(c) dipping the stamp in a natural or synthetic polymer-based ink;

(d) removing any excess polymer-based ink from the stamp;

(e) placing the stamp in contact with a surface of the polymeric substrate to be patterned so that the natural or synthetic polymer transfers from the stamp to the substrate and produces a pattern of micron-sized features on the polymeric substrate; and

(f) removing the stamp from the surface of the polymeric substrate.

Preferred embodiments of the present invention also include a polymeric substrate having a surface patterned with micron-sized features, wherein the surface of the polymeric substrate is patterned in accordance with the present methods, and devices for guided tissue regeneration which include such a polymeric substrate. The polymeric substrate preferably is a biocompatible polymer.

### BRIEF DESCRIPTION OF THE DRAWINGS

FIG. 1 depicts a general method for making an article of the present invention. FIG. 1A provides a schematic diagram of a stamp where the pre-selected pattern is represented by raised features with particular shapes, in this case the pattern is a series of lines which, when viewed in cross-section, appear to be rectangles projecting from the body of the stamp. FIG. 1B provides a schematic diagram of a polymeric substrate. According to the present invention, the polymeric substrate (FIG. 1B) is activated by plasma to generate a transiently polarized surface which can stably bind biologically active molecules. The stamp (FIG. 1A) may also be plasma—treated to facilitate transfer of the biologically active molecules. FIG. 1C depicts the stamp coated with a solution of biologically active molecules (wiggly lines). Excess solution is removed from the stamp and the polymeric substrate is stamped as shown in FIG. 1D, to transfer a pre-selected pattern of biologically active molecules to the polymeric substrate. FIG. 1E depicts the polymeric surface with the pattern of biologically active molecules after stamping.

FIG. 2 is a photomicrograph illustrating a pattern of biologically active molecules (laminin) stably adsorbed on the surface of a polymeric substrate. In this case the pattern is a series of lines. To permit visualization, the stably adsorbed laminin has been exposed to a solution of fluorescently tagged anti-laminin antibodies. After washing off non-specifically bound antibodies, the bound antibodies were observed under fluorescent rumination.

FIG. 3 is a photomicrograph illustrating cellular adhesion and growth on stably adsorbed laminin where the laminin has been adsorbed in no pattern (left) and in a pattern consisting of a series of lines (right).

FIG. 4 is a photomicrograph depicting the pattern of neurite outgrowth from neuronal cells. Neuronal cells were plated onto a pattern of laminin consisting of a series of lines. An optical microscope was used to visualize the pattern of neuronal process outgrowth from the adhered cells. As illustrated, most neuronal processes adhere to and grow along the lines of the laminin pattern.

### DETAILED DESCRIPTION OF THE INVENTION

Applicants have discovered methods for treating a hydrophobic polymeric substrate to make its surface transiently polar so that biologically active molecules can be directly and stably adsorbed onto that surface. According to the present invention, such treatment does not substantially alter the chemical composition of the polymeric substrate but does make the polymeric substrate polar in nature for a sufficient time to apply a pre-selected pattern of biologically active molecules. Despite the absence of a covalent linkage, biologically active molecules adsorbed onto a polymeric substrate by the present methods are surprisingly stable. The functional groups and active sites of adsorbed biologically active molecules also remain free to interact with other molecular species, macromolecules, biomolecules, cells and tissues.

An article made from a polymeric substrate with a stably adsorbed pattern of biologically active molecules exhibits many advantages. One advantage of adsorption is that linker molecules between the polymeric substrate and the biologically active molecules are not needed. A second advantage is that the article has fewer components and will generate fewer immunogenic responses and fewer side effects. A third advantage is that manufacturing procedures are simple, fast, inexpensive and avoid coupling agents and other harsh chemicals which may create negative side effects when left in the article in even small amounts. There is no need for UV exposure, high processing temperatures, organic solvents, expensive equipment, extensive manufacturing space and/or lengthy production times. Finally, the present methods produce a polymeric substrate surface which is only transiently polar and which can return to being hydrophobic. Hence, articles with patterns of biologically active molecules that are surrounded by regions of hydrophobicity tend to encourage specific interaction of biomolecules and cells with the stably adsorbed biologically active molecules. Nonspecific binding of biomolecules and cells to the unbound surface of the polymeric substrate is discouraged because that surface is hydrophobic.

### Definitions

The term “biologically active molecule” is used herein to denote a molecule that can be stably adsorbed on the surface of the polymeric substrate of the present inventive articles and which has a useful in vivo or in vitro function. In general, such biologically active molecules can have an effect on a biological process such as cell adhesion, cell growth, cell-to-cell contact or communication and the like. In addition, the present biologically active molecules stably adsorb to the polymeric substrate by nonspecific molecular interactions. Typically, portions of the biologically active molecules will be polar or somewhat hydrophilic in nature so that they can adsorb into the plasma-activated polymeric substrate. Hence, biologically active molecules having functional groups which have a dipole moment, such as amines, amides, carbonyls, carboxylates, esters, alcohols, sulfhydryls and the like are particularly suited for use in the present invention. Examples of biologically active molecules for use in the present invention include hormones, extracellular matrix molecules, cell adhesion molecules, natural polymers, enzymes, peptides, antibodies, antigens, polynucleotides, growth factors, synthetic polymers, polylysine, drugs and other molecules. Additional specific examples are provided below.

As used herein, the term “hydrophilic” refers to a general affinity for a chemical group to attract water or to otherwise exhibit sufficient polarity to permit stable adsorption to another polar group. The term can be used to describe chemical groups in both the polymeric substrate and the biologically active molecules and includes groups such as amines, amides, carbonyls, carboxylates, esters, alcohols, sulfhydryls and the like. However, as provided herein, the surface of the activated polymeric substrate does not become so hydrophilic as to permit diffusion of the biologically active molecules during stamping or application of the pre-selected pattern.

The term “micron-sized,” as used herein, means that a pattern or article is microscopic in size. In general, micron-sized patterns and articles are about 10 nm to about 1000 microns in size. Preferably micron-sized patterns are about 0.1 microns to about 500 microns in size. More preferably, micron-sized patterns are about 1 micron to about 100 microns in size. Most preferred patterns and articles are about 1 micron to about 50 microns in size.

As used herein, a “plasma” is an ionic gas capable of making a polymeric substrate transiently polar. Any such plasma known to one of skill in the art can be used in the present invention. Examples of plasmas which can be used include argon, nitrogen, oxygen, and other plasmas.

The term “polar,” as used herein, refers to groups on the surface of the polymeric substrate and on the biologically active molecules of the invention which have a dipole moment. For example, the polymeric substrate may be made polar by modifying the surface energy or surface tension of organic groups within the polymeric substrate or by temporarily aligning the dipole moments or polarity of the polymeric molecules within the substrate. The term can be used to describe chemical groups in the polymeric substrate and in the biologically active molecules and includes groups such as amines, amides, carbonyls, carboxylates, esters, alcohols, sulfhydryls and the like.

As provided herein, the term “polymeric substrate” includes polymers which have sufficient mechanical stability and strength to serve as the structural material of an article of the present invention and which are capable of becoming transiently polar when treated with an ionic gaseous plasma according to the methods of the present invention. Non-polymeric substances, such as glass and silicone, are not contemplated as polymeric substrates by the present invention. Generally, the present polymeric substrates are hydrophobic before treatment with an ionized plasma and become sufficiently polar to adsorb biologically active molecules when treated with an ionic gaseous plasma. However, after such treatment the surface of the present polymeric substrate will return to its original state, and become more hydrophobic unless that surface has adsorbed biologically active molecules. Polymers selected for use in the present articles are preferably biocompatible and are preferably not polydimethylsiloxane. Examples of polymers for use in the present invention are provided below.

As used herein, a “pre-selected pattern” is a shape of biologically active molecules useful for adhesion, detection, growth or identification of other molecular species, macromolecules, biomolecules, cells and tissues. Hence, while a mixture of biologically active molecules will adopt a “pre-selected pattern,” other molecular species, macromolecules, biomolecules, cells and tissues which bind to or interact with those biologically active molecules need not initially adopt that pattern. Instead, such molecular species, macromolecules, biomolecules, cells and tissues are only encouraged to grow into or otherwise adopt such a pre-selected pattern, for example, as time progresses. Pre-selected patterns can be any pattern contemplated by one of skill in the art, including lines, circles, ovals, squares, rectangles, diamonds, triangles or a combination of any of these shapes. In general, the pre-selected pattern on a polymeric substrate is not a raised pattern but such a pattern can be a flat or curved planar surface.

As provided herein, the term “stably adsorbed” means that the biologically active molecules are bound to the surface of the polymeric substrate by nonspecific molecular interaction. Stably adsorbed biologically active molecules can readily be stamped or applied to the surface of the polymeric substrate without significant diffusion to form a crisp, distinct pattern, which accurately reflects the pre-selected pattern of the stamp. Biologically active molecules which are stably adsorbed to the polymeric substrates of the present invention will remain adsorbed for a sufficient time and with a sufficient affinity to be used for the methods of the present invention.

As used herein, a “stamp” is an object used for contacting and thereby transferring a pre-selected pattern of biologically active molecules to a surface of the inventive articles. In general, a stamp has a pre-selected pattern physically raised from its surface. According to the present invention, a stamp is distinct from an article of the present invention.

The term “stamping” as used herein refers to any procedure for transferring a pre-selected pattern of biologically active molecules from a stamp to an article having a polymeric substrate.

### An Article having a Pattern of Biologically Active Molecules

The present invention provides an article having a pre-selected pattern of biologically active molecules stably adsorbed directly onto a polymeric substrate. The pattern typically includes a bound polymeric substrate surface having biologically active molecules stably adsorbed thereto and an unbound polymeric substrate surface having substantially no biologically active molecules adsorbed thereto. While any size article and pattern is contemplated, micron-sized articles and patterns are preferred. According to the present invention, the article is not a stamp that is used to create a pattern. Instead, the present articles have a final pattern of biologically active molecules stably adsorbed to their surfaces which can now be used for a variety of purposes including biological testing and assays, tissue regeneration, micropatterning and directing the spatial alignment of biomolecules and cells which adhere to the biologically active molecules.

The biologically active molecules are stably adsorbed directly onto a polymeric substrate by nonspecific molecular interaction. According to the present invention, such stable adsorption by nonspecific molecular interaction is accomplished by making the surface of the polymeric substrate polar so that the biologically active molecules can be adsorbed to it. While the polymeric substrate is made sufficiently polar to permit stable adsorption of biologically active molecules, it is not made so polar that such biologically active molecules will diffuse across the surface of the polymeric substrate during application of the pre-selected pattern. In a preferred embodiment, the surface of the polymeric substrate is hydrophobic but is activated to become somewhat transiently polar before the adsorption process. Portions of the polymeric substrate which do not adsorb biologically active molecules preferably return to their hydrophobic state after adsorption is complete.

The surface of the polymeric substrate can have any shape, for example, it can be flat, curved or tubular. In general, the surface of the polymeric substrate is not raised; instead, it is preferably a flat or curved planar surface. For purposes of this invention, the polymeric substrate can be biodegradable or nondegradable. Typically, to be useful in both in vivo and in vitro applications, the polymeric substrates of the present invention are nontoxic, biocompatible, processable, transparent for microscopic analysis and mechanically stable.

### Polymers for Use with the Invention

The main criteria used to select a polymer for use with the present invention is that the polymer is capable of becoming transiently activated toward adsorption of the biologically active molecules of the present invention (e.g., the surface of the polymer is made transiently polar) when treated with an ionic gaseous plasma according to the methods of the present invention. The present polymeric substrates are generally hydrophobic before treatment with an ionized plasma and, unless adsorbed to biologically active molecules, will return to being hydrophobic after the adsorption process is completed. Preferably, the polymeric substrate is not polydimethylsiloxane. Polymers selected for use in the present articles are preferably biocompatible, meaning that the polymers and their degradation products are not unacceptably immunogenic, allergenic or toxic.

A large variety of polymers may be used as substrates in the articles of the present invention. One of skill in the art can readily select an appropriate polymeric substrate with sufficient mechanical stability and with sufficient transient polarity to be used in the methods of the present invention. Examples of polymers useful for the present articles and methods include polyacrylates, polymethylacrylates, polycarbonates, polystyrenes, polysulphones, polyhydroxy acids, polyanhydrides, polyorthoesters, polyphosphazenes, polyphosphates, polyesters, nylons or mixtures thereof. Examples of polymers of polyhydroxy acids include polyhydroxybutyric acid, polylactic, polyglycolic and polycaproic acid. Polyanhydrides, polyorthoesters, polyphosphazenes, polyphosphates, polycaprolactone or copolymers prepared from the monomers of these polymers (see for example WO 95/03357) may also be used. Poly(ortho esters), polyol/diketene acetals and related polymers are provided by Heller, ACS Symposium Series 567, 292-305, 1994. Examples of biodegradable hydrophobic polyanhydrides are disclosed, for example, in U.S. Pat. No. 4,757,128; U.S. Pat. No. 4,857,311; U.S. Pat. No. 4,888,176 and U.S. Pat. No. 4,789,724. Polyhydroxybutyrates are disclosed in U.S. Pat. No. 3,044,942.

Polymers of lactic acid or glycolic acid, or copolymers of these monomers are contemplated, such as polylactic acid, polyglycolic acid or poly(lactic-co glycolic) acid., poly(E-caprolactone), poly(3-hydroxybutyrate), poly(p-dioxanone), polyalkylene glycol, polyethylene and polypropylene fumarate.

Polyanhydrides for use as the polymeric substrate of the present invention include, but are not limited to: poly(sebacic anhydride), poly(carboxybiscarboxyphenoxyphenoxyhexane), poly [bis(p-carboxyphenoxy) methane], and copolymers thereof which are described by Tamada and Langer in Journal of BioMaterials Science Polymer Edition, 3:315 (1992) and by Domb in Chapter 8 of the Handbook of Biodegradable Polymers, ed. Domb A. J. and Wiseman R. M., Harwood Academic Publishers. Also contemplated are poly(amino acids), and poly(pseudo amino acids) that include those described by James and Kohn in pages 389-403 of Controlled Drug Delivery Challenges and Strategies, American Chemical Society, Washington D.C. Polyphosphazenes for use in the present invention include derivatives of poly[(dichloro) phosphazene] poly[(organo) phosphazenes] polymers described by Schacht in Biotechnology and Bioengineering, 52, 102-108, 1996.

In a preferred embodiment, polyesters of poly(lactic-co-glycolic)acid (“PLGA”) are used. These polymers are approved for parenteral administration by the FDA. Because PLGA degrades via non-enzymatic hydrolysis in the initial stages, in vivo degradation rates can be predicted from in vitro data. PLGA is also a desirable substrate because it degrades to lactic and glycolic acids, substances found naturally in the body.

Additionally, copolymers with amino acids are contemplated as polymeric substrates of the present invention, for example, glycolic acid and glycine, or lactic acid and lysine as described in Barrera et al., J. Am. Chem. Soc., 115:11010 (1993) and Cook et al., J. Biomed. Mat. Res., 35:513 (1997). Biodegradable materials also include collagen and polysaccharide gels, for example, of hyaluronic acid which may be altered by chemical modification to alter its polarity. Copolymers of collagen and proteoglycans may also be used.

Protein polymers may also be used for the polymeric substrate and are prepared by available protein chemistry and molecular biology techniques. For example, polymers based on silk or elastin repeating units may be prepared and are suitable for use in the present invention (Hubbell J A., Biotechnology, 13:565 (1995)). It will be appreciated that some biocompatible polymers, for example, some natural polymers as described above, may degrade in response to cellular and enzymatic activity and that the rate of such degradation may vary depending on the environment or cultural conditions involved. While such degradation can be a useful property, particularly when the polymeric substrate is used in vivo, the rate of degradation by the polymeric substrate will preferably not be faster than the rate of tissue regeneration or the time needed for analytical testing. The rate of degradation in a specific environment can be observed by methods known to one of skill in the art. For example, the rate of degradation can be observed by placing the polymeric substrate in the environment in which it will be used and observed how long it remains intact. Hence, degradation can readily be observed and manipulated by one of skill in the art.

### Biologically Active Molecules for Use with the Invention

The term “biologically active molecule” is used herein to denote a molecule that can be stably adsorbed on the surface of the polymeric substrate of the present inventive articles which has a useful in vivo or in vitro function. Biologically active molecule therefore include any molecule that can effect a biological process, such as cellular adhesion, growth or differentiation. Biologically active molecules that inhibit or promote growth and/or differentiation of a particular type of cell are contemplated. It is preferred that the biologically active molecule is a peptide, protein, carbohydrate, nucleic acid, lipid, polysaccharide, or combinations thereof, for example a proteoglycan, or synthetic inorganic or organic molecule. Examples of biologically active molecules for use in the present invention include hormones, extracellular matrix molecules, cell adhesion molecules, natural polymers, enzymes, peptides, antibodies, antigens, polynucleotides, growth factors, synthetic polymers, polylysine, drugs and other molecules.

The following biologically active molecules are understood to be exemplary and are not to be limiting in any manner. Examples of biologically active molecules that may be used include cell binding domains of extracellular matrix proteins and other adhesion proteins, for example fibronectin and vitronectin, or fragments thereof, that are recognized by cytoskeleton associated receptors in the cell membrane, known as integrins. Such receptors can bind to a small domain on the adhesion proteins, for example, the peptide sequence Arg-Gly-Asp (also referred to a “RGD”), which is found in many adhesion proteins, and which binds to many integrins. Varying the sequence or flanking sequences can alter the binding affinity of a receptor for the peptide or protein containing it. The density of the biologically active molecule in the pre-selected pattern may affect adhesion, binding and cellular responses, and it will be appreciated that it may be necessary to control the density of the biologically active molecule to get the optimum density for practicing the present methods.

Further examples of biologically active molecules contemplated by the present invention include the peptide Tyr-Ile-Gly-Ser-Arg (SEQ ID NO:1), found in the B1 chain of laminin which binds to the 67 kDa laminin receptor found on many cell types, and the peptide Ile-Lys-Val-Ala-Val (SEQ ID NO:2) found in the A chain of laminin which binds a 110 kDa receptor and which can induce neurite growth. Many different peptides with SEQ ID NO:2 sequence may stimulate neurite extension and any peptide that includes a sequence of amino acids that is able to bind to a cell adhesion receptor is contemplated by the present invention. For example, the isolated SEQ ID NO:2 peptide may not be sufficiently water soluble for all of the present applications. As an alternative, the water soluble peptide Cys-Ser-Arg-Ala-Arg-Lys-Gln-Ala-Ala-Ser-Ile-Lys-Val-Ala-Val-Ser-Ala-Asp-Arg (SEQ ID NO:3) may be used. The peptide Arg-Glu-Asp-Val (SEQ ID NO:4) from fibronectin binds to the integrin on human endothelial cells, but does not support adhesion or spreading of smooth muscle cells, fibroblasts or platelets and may therefore be useful for achieving selective cell adhesion.

Cell binding domain sequences of extracellular matrix proteins may also be used as biologically active molecules within the present invention. Examples of such domain sequences include: the Arg-Gly-Asp-Ser (SEQ ID NO:5) peptide sequence found in fibronectin which can mediate adhesion of most cells via the ap receptor; the Leu-Asp-Val and Arg-Glu-Asp-Val (SEQ ID NO:6) peptide sequences from fibronectin which can also mediate adhesion of cells; the Arg-Gly-Asp-Val (SEQ ID NO:7) peptide sequence from vitronectin which can mediate adhesion of most cell types via the ccp receptor; the Leu-Arg-Gly-Asp-Asn (SEQ ID NO:8) peptide sequence from Laminin A which can mediate cell adhesion; the Pro-Asp-Ser-Gly-Arg (SEQ ID NO:9) peptide from Laminin B1 which can mediate cell adhesion; the Arg-Asn-Ile-Ala-Glu-Ile-Ile-Lys (SEQ ID NO:13) peptide from Laminin B2 which can mediate neurite extension; the short Asp-Ala dipeptide sequence; the Arg-Gly-Asp-Thr (SEQ ID NO:10) peptide from Collagen I which can mediate adhesion of most cells; the Asp-Gly-Glu-Ala (SEQ ID NO:11) sequence which can mediate adhesion of platelets and other cells; and the Val-Thr-Xaa-Gly (SEQ ID NO:12) of thrombospondin which can mediate adhesion of platelets.

Further examples of biologically active molecules useful in the present invention include epidermal growth factor, nerve growth factor, insulin-like growth factor, basic fibroblast growth factor, platelet derived growth factor, transforming growth factor and related growth factors. Other examples include bone morphogenetic proteins, cytokines including interferons, interleukins, and monocyte chemotactic protein-1. It will be appreciated that the biologically active molecules of the present invention may also be provided on biocompatible, biodegradable polymeric substrates and so that they may be released as the material degrades.

Further examples of biologically active molecules contemplated by the present invention include dopamine, amine-rich oligopeptides, such as heparin binding domains found in adhesion proteins such as fibronectin and laminin. Other examples include amines, basic amino acids, and monosaccharides which can bind to the asialoglycoprotein receptor on hepatocytes. For example, one can stably adsorb N-acetylglucosamine or lactose or a polymerized N-acetyllactosamine monomer to the polymeric substrates of the present invention. Another example is sialyl Lewis X saccharide (Varki, Proc. Natl. Acad. Sci., (USA) 91:7390 (1994)) which is a biologically active molecule for the selectin class of saccharide-binding receptors that are usually responsible for mediating cell-cell interactions (Lasky, Science, 258:964 (1992)). Thus this saccharide may be useful for mimicking cell-cell recognition.

Bone morphogenetic proteins may be useful as biologically active molecules of the present invention for closure of defects in bone. Basic fibroblast growth factor is a useful biologically active molecule for inducing vascularisation. A slow release formulation, wherein the biologically active molecules are slowly released from the degrading polymer may be effective for these molecules.

The suitability of a biologically active molecule for use in the present invention may be assessed by methods known to those skilled in the art. For example, when it is desired to bind a specific biomolecule or cell to an article of the present invention, a potential biologically active molecule is stably adsorbed to a polymeric substrate and the binding or adhesion of a biomolecule or cell can be assessed by observing whether the cell or biomolecule binds to a polymeric substrate, by measuring protein-protein interactions between the biomolecule or cell and the biologically active molecules, or by detecting whether an antibody reactive with the biomolecule or cell becomes bound to the polymeric substrate. Functional assays for detecting whether a cell responds to a biologically active molecule may also be used, for example, functional assays capable of assessing whether the cell grows or adheres to the biologically active molecule under consideration. All of these procedures are available and can be adapted by one of skill in the art to identify biologically active molecules which are suitable for use in the present articles and methods.

### Methods for Preparing Articles with Patterns of Biologically Active Molecules

The invention provides methods to prepare an article containing a polymeric substrate onto which one or more biologically active molecules are stably adsorbed through a non-specific molecular interaction. The method involves treating the surface of a polymeric substrate to increase the ability of the surface to stably adsorb the biologically active molecules. A type or mixture of biologically active molecules are then transferred to the surface of the substrate where they are directly and stably adsorbed to the polymeric substrate through a polar interaction between the surface and the biologically active molecule.

The surface of the substrate is treated prior to contact with the biologically active molecule to increase the polarity of the surface. In order to accomplish this, the polymeric substrate is exposed to conditions which increase polarity of the surface of the polymeric substrate. Any method known to one of skill in the art can be used to increase the polarity of the surface of the polymeric substrate. In preferred embodiments, the polymeric substrate may be made polar by modifying the surface energy or surface tension of organic groups within the polymeric substrate or by temporarily aligning the dipole moments or polarity of the polymeric molecules within the substrate. Polymers having polar groups such as amines, amides, carbonyls, carboxylates, esters, alcohols, sulfhydryls and the like may be preferred substrates for incorporation into the articles of the present invention.

In one embodiment, the polymeric substrate is placed in a low temperature plasma generator and exposed to an ionized gas plasma for a time and at a temperature and pressure which temporarily increase the polarity of surface of the polymeric substrate. For example, the polymeric substrate can be exposed to a stream of plasma for a temperature and under an electrical wattage sufficient to make the surface of the polymeric substrate polar. The rate of exposure to plasma can vary depending on the type of plasma, the temperature and other factors. For example, a rate of about 0.01 to 100 cc/minute, preferably about 0.1 to 50 cc/minute and more preferably about 1-10 cc/minute can be used One of skill in the art can also vary the time of such exposure as needed to make the surface of the polymeric substrate polar. For example, the time of exposure to plasma can include any suitable time, such as for example from about 0.5 to 300 seconds, preferably from about 1 to 200 seconds and more preferably from about 5-120 seconds. One of skill in the art can also readily determine a temperature sufficient to make the surface of the polymeric substrate polar. For example, convenient temperatures for making the polymeric substrate polar are about 5° C. to about 42° C., preferably about 10° C. to about 37° C. and more preferably about room temperature. An electrical wattage sufficient to make the surface of the polymeric substrate polar varies with the type of gaseous plasma. For example, such a wattage can vary from about 5 to 500 Watts, preferably about 50-400 Watts and more preferably about 100-300 Watts. Convenient conditions for making the surface of the polymeric substrate polar include exposing the article to oxygen plasma at a rate of about 4 cc/minute, for about 30 seconds at 200 Watts and at room temperature.

Any ionic gaseous plasma known by one of skill in the art to activate the surface of the polymeric substrate can be used. Types of plasmas contemplated by the present invention include argon, nitrogen, oxygen, and other gases known to those of skill in the art to readily be ionized. Preferably the surface is treated with oxygen plasma.

Polymeric substrates treated in the manner describe were evaluated via x-ray photoelectron spectroscopy, atomic force microscopy, and near-field scanning optical microscopy. When using the present methods to make the surface of the polymeric substrate polar, it was observed that the surface was not significantly chemically changed. While such treatment does not substantially alter the chemical composition of the polymeric substrate, it does make the polymeric substrate sufficiently polar in nature for adsorption of a defined, pre-selected pattern of biologically active molecules. Moreover, such treatment does not make the polymeric substrate so hydrophilic or so polar that the applied pattern of biologically active molecules becomes obscured by diffusion. Such treatment also transiently activates or polarizes the surface of the polymeric substrate for a sufficient time to apply a pre-selected pattern of biologically active molecules, for example, for about 5 to about 60 minutes, after which time the substrate will return to its original state.

Transferring a pre-selected pattern of biologically active molecules to the surface of a polymeric substrate generally involves contacting that surface with one or more biologically active molecules such that the biologically active molecules are retained on the surface of the substrate in a pre-selected pattern through a non-specific molecular interaction. Any method used by one of skill in the art to apply a pattern of biologically active molecules to a polymeric substrate can be used. For example, biologically active molecules can be applied using a roller, printer, stamp or similar apparatus. According to the present invention, all of these methods are termed “stamping” the biologically active molecules onto the polymeric substrate. Stamping methods of the present invention allow for production of multiple micropatterned polymeric substrates in minimal amounts of time (e.g. minutes). In contrast, preparation of multiple micropatterned polymeric substrates via prior art microlithography can take several days or even weeks.

In order to transfer a pattern of biologically active molecules to a polymeric substrate, the stamp is coated with biologically active molecules. However, only biologically active molecules in the stamp pattern are transferred to the substrate. Preferably the stamp pattern is a raised pattern, but any method known to one of skill in the art for making a pattern on a stamp is contemplated. Preferably the pattern is micron-sized. Upon pressing the stamp to the surface of the polymeric substrate, the pattern of biologically active molecules is transferred to the polymeric substrate. Hence, the biologically active molecules are stamped onto the surface of the substrate in a spatially controlled manner. The micron-sized pattern can be any pattern contemplated by one of skill in the art, including a line, circle, oval, square, rectangle, diamond, triangle or a combination of of these shapes. Selection of a pattern is dependent upon the application which the article is intended to be used. For example, in one embodiment the pattern is a line. Biomolecules and cells may adhere to the biologically active molecules in the shape of a line so that a linear pattern of cells can be formed or so that cells placed at one end of the line will grow toward the other end of the line. The line can have any width of use to one of skill in the art. For example, for micron-sized patterns, the line can be about 0.01 to 10 microns in width, and about 10 to 500 microns in length. Preferably, such lines are about 0.1 to 1.0 microns in width and about 1 to 100 microns in length. More preferred line patterns are about 5 micron to about 50 microns in size.

As described above, a stamp may be used to contact the surface of the polymeric substrate with the biologically active molecules. The stamp is dipped in a natural or synthetic solution of the biologically active molecules. A preferred vehicle for the biologically active molecules is Hank's buffered saline solution or the like. The stamp is then placed in contact with the surface of the polymeric substrate to be patterned and left for approximately 1-120 minutes, preferably about 15 minutes, so that the biologically active molecule transfers from the stamp to the substrate. The stamp is then removed and the patterned substrate can be used immediately or stored in a saline solution for future use.

A stamp use for in the present invention can be made by any method known to one of skill in the art. One procedure for making a stamp involves the preparation of a master that has the reverse image of the micron-sized pattern to be placed on the stamp. Briefly, a master may be fabricated on a polished silicon wafer using AS P4620 photoresist (Clariant, Inc.) which is spin coated to a thickness of about 5 mm and processed by contact photolithography. Methods for the production of masters are known in the art. See, Moread, W. M., Semiconductor Lithography: Principles and Materials, Plenum, New York (1988); Brambley et al., Adv. Mater. Opt. Electron., 4:55 (1994); Handbook of Microlithography, Micromachining, and Microfabrication, Vol. 1 (Ed: P. Rai-Choudbury), SPEI Optical Engineering Press, Bellingham, Wash. (1997).

Any material known to one of skill in the art can be used to make the stamp. A preferred material for the stamp is poly(dimethyl siloxane) (PDMS). One source of PDMS material is Sylgard 184™ (Dow Corning, Midland, Mich.). The flexibility of a stamp prepared from this material permits patterning of non-flat surfaces such as round or cylindrical polymeric substrates. For PDMS stamp preparation, the PDMS material is first prepared in liquid form and mixed with the curing agent. Bubbles are removed from the mixture, preferably via a vacuum at 28″ Hg, and the mixture is poured over a master pattern. It is preferred that the master pattern comprise an organic or inorganic material such as glass which remains hard at temperatures greater than 60° C. Any bubbles are again removed preferably via a vacuum. This mixture is then permitted to cure at approximately 60° C. for a minimum of 4 hours. The resulting stamp is released from the master pattern upon curing. The stamp can also be treated with a plasma as described above to make it more hydrophilic or polar so that the selected biologically active molecules will adsorb to it.

### Methods for Spatially Direct Cellular Growth with the Present Articles

The invention also provides methods for using the present articles for several purposes including modulating cell-substrate interactions, spatially directing cell growth, tissue regeneration, combinatorial screening strategies, and multiple analytical biosensors. The present methods allow the growth of the cell to be modulated through adhesion and/or growth stimulation along the specific pattern of biologically active molecules on the surface of the article. Patterned polymeric devices prepared in accordance with the methods of this invention are particularly useful in tissue engineering applications where the present micropatterned polymeric substrates act as templates to guide and regulate cell growth after promoting cellular adhesion during tissue engineering applications.

The present invention therefore provides methods for spatially modulating the growth of a cell which include contacting a cell with an article of the present invention for a time and under conditions sufficient to adhere the cell to the biologically active molecules on the article and to grow the cell along the micron-sized pattern of biologically active molecules on the polymeric substrate of the article. This method is used on any mammal, preferably a human. Any cell type known to one of skill in the art may be used in the present methods so long as a biologically active molecule can be found which will interact or bind to that cell type. Examples of cell types which can be used in the present methods include nerve cells, epithelial cells, mesenchymal stem cells, fibroblast cells, and other cell types. In some instances, stem cells are preferred. In a preferred embodiment the cell is a nerve cell.

An article of the present invention is thus employed as a tissue regeneration template. The articles of the invention may be used in any mammal. However, it is preferred that the mammal is a human and that the article is used in medicine. In order to regenerate tissue into a desired shape, mammalian tissues are encouraged to grow along the patterned lines and shapes of adsorbed biologically active molecules. The present methods involve contacting the surface of the article of the invention with a cell such that the cell adheres to a biologically active molecule provided on the surface of the article. Conditions sufficient to adhere the cell to the biologically active molecules on the article and to grow the cell along the micron-sized pattern of biologically active molecules on the polymeric substrate of the article include cell culture conditions and conditions permitting cell growth. The cell can be contacted with the article in vitro or in vivo. Preferably the cell is contacted with the article in vivo by implanting the article into a specific site. The site chosen can be any site but the present methods are often used to repair injuries, help regenerate tissues and directionally guide cellular growth. Any tissue type can be treated, including skin, vascular, and neural tissues. Preferably the tissue is a neural tissue.

According to the present invention, molecular interactions between neurons and the pattern of biologically active molecules encourage neurite extension. Articles prepared according to the invention with particular cell adhesion proteins, growth factors and other biologically active molecules is beneficial in promoting such neurite extension. Such an article can be patterned as a hollow tube of polymer with biologically active molecules that promote neurite extension adsorbed inside or outside the tube. Such tissue engineering can be initiated outside the body by, for example, removing cells from a patient and seeding those cells onto the article in an appropriate culture medium. When the cells have grown, divided and/or differentiated to form a tissue in culture, the new tissue may be implanted into the body. The article may be implanted at any stage in the growth of the tissue, depending on clinical need and one of skill in the art can readily determine when implantation is appropriate. A biodegradable polymeric substrate in the article can be removed by hydrolysis and dissolution in culture before the engineered tissue is implanted into the patient if the function of the substrate is complete. Thus, the polymeric substrate can be designed to be completely degraded during in vitro culture or it can be designed to provide support to the bioengineered tissue (for example, a nerve) for a substantially longer predetermined period after surgical implantation.

Two examples of tissue engineering applications in which the present methods may be used are directed nerve regeneration and new blood vessel formation (vasculogenesis). For nerve regeneration applications, patterns composed of the biologically active molecules which include peptide sequence Ile-Lys-Val-Ala-Val (SEQ ID NO:2) may be used to encourage nerve cell growth to follow predetermined pathways, i.e. between two severed points of a nerve or towards a de-nerved tissue. For vasculogenesis applications, endothelial cells can be encouraged to grow along patterns of biologically active molecules which include the Arg-Gly-Asp peptide sequence.

A method of forming an article for regenerating tissue according to the present invention may be carried out substantially as described below. First, a biocompatible polymeric substrate is chosen which is optionally be biodegradable over a desired time period. Second, a type of biologically active molecules or a mixture of biologically active molecules is selected which will provide the desired functions, for example, cell adhesion and/or cell growth. Third, a spatially controlled pattern of the selected biologically active molecules is stamped on or applied to an activated surface of the polymeric substrate. In order to regenerate a tissue, the article can be surgically implanted at the appropriate site or cultured in vitro with surgically removed cells and then surgically implanted, as described above.

An advantage of the present articles is that the non-coated surface of the substrate can be hydrophobic. This property decreases the ability of cells and other biomolecules to adhere to the non-covered regions of the polymeric substrate. Hence, cells adhere more specifically to the regions of the polymeric coated by biologically active molecules and the article is therefore better able to direct cell growth. Another advantage of the invention is that cells may be grown in vitro under common laboratory conditions or in vivo upon implantation of the article into a living creature.

Articles comprising the patterned polymeric surfaces prepared via the method of the present invention therefore have various uses. For example, directed growth of neurons is necessary for the repair of peripheral nerve damage. When injury occurs to the nerve, regrowth of the affected axons must be directed along their original path for function to resume. In the body, both physical and chemical cues direct regrowth. Substrates prepared in accordance with the process of the present invention can mimic some of these cues thereby encouraging nerve cell alignment and regrowth.

The following nonlimiting examples are provided to further illustrate the invention.

### EXAMPLES

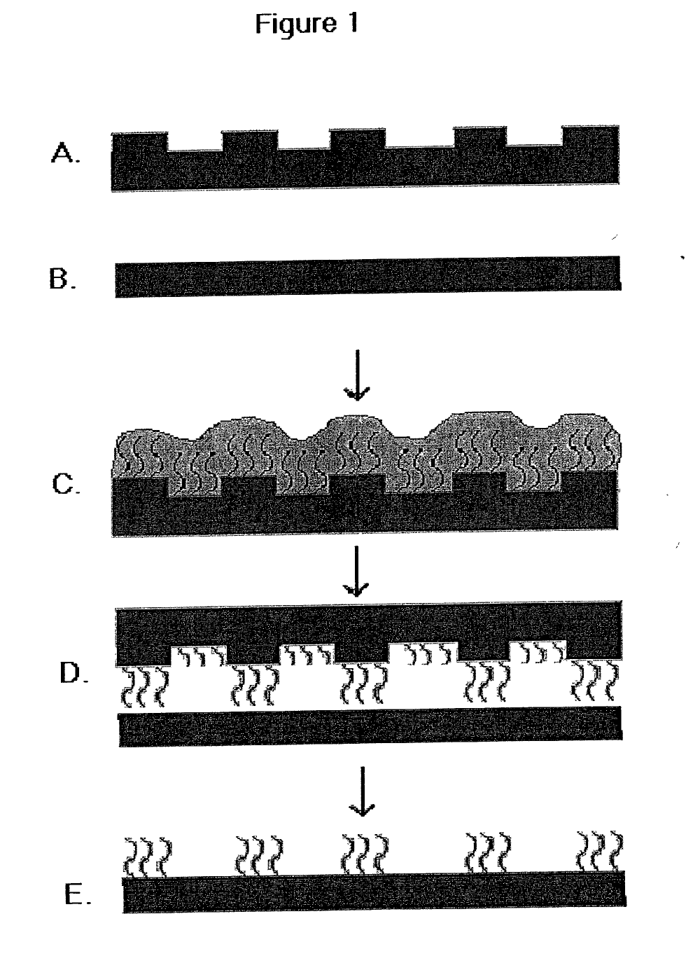

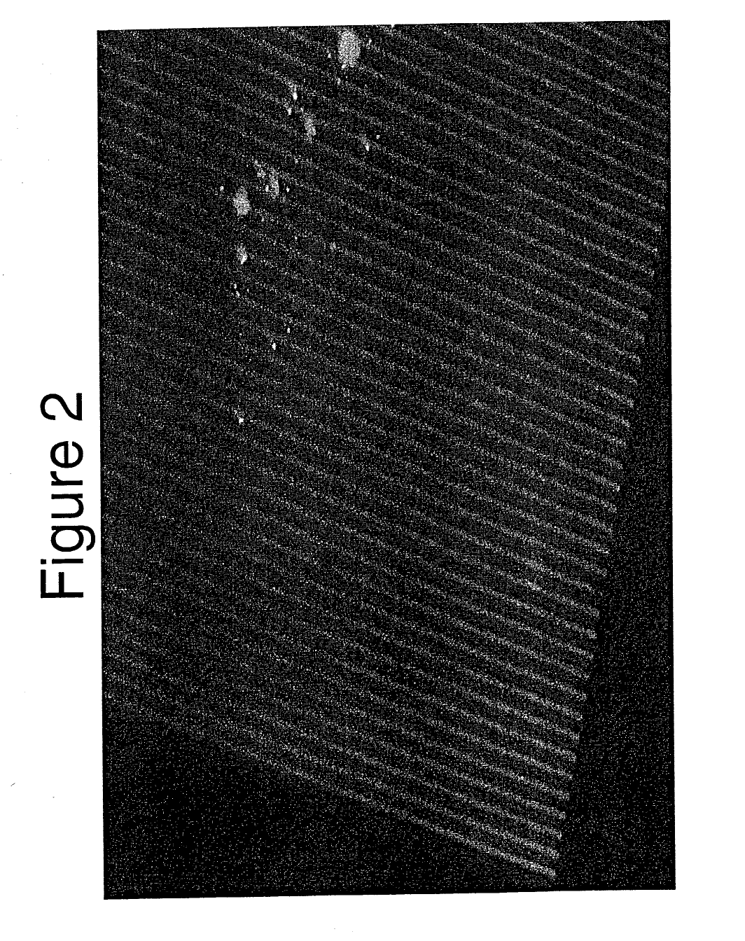

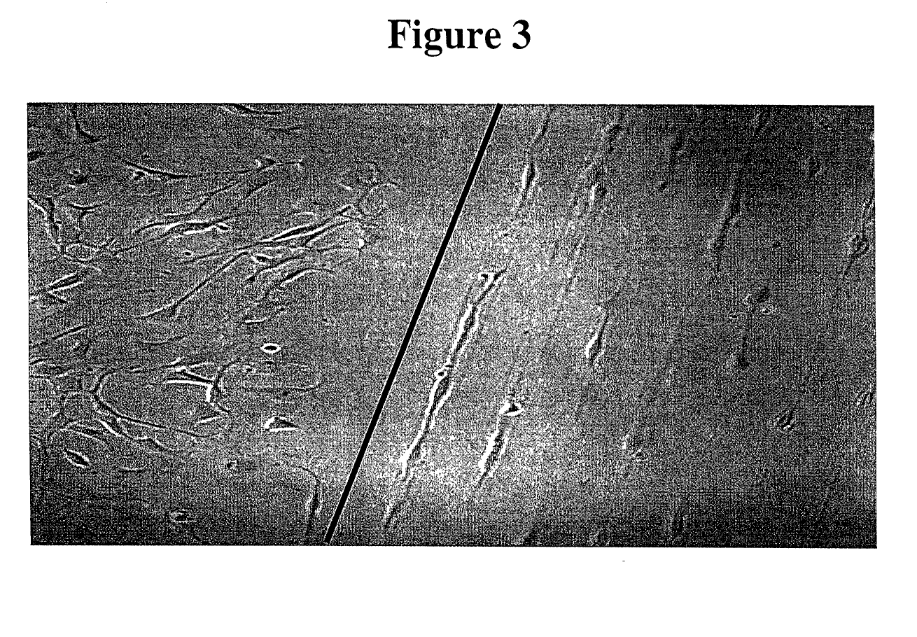

A nondegradable polymer, poly(methyl acrylate) (PMMA, MW 120,000, Aldrich) and a degradable polymer, poly(lactide-co-glycolide) (Medisorb) were evaluated as polymeric substrates for micropatterning via the microcontact process of the present invention. See FIG. 1 for the general procedure. For these experiments, the polymers were compressed into thin films of 200 μm and cut to the size of coverslips. After activation, polymeric substrates were evaluated via x-ray photoelectron spectroscopy, atomic force microscopy, and near-field scanning optical microscopy, and it was observed that the polymeric surface was not significantly changed. After laminin absorption, its deposition was determined by incubating the prepared slips with rabbit anti-laminin affinity isolated antibody and FITC-conjugated goat anti-rabbit IgG (see FIG. 2). Dorsal root ganglia dissected from seven day old chick embryos were either directly plated onto the patterned substrate or dissociated into individual neurons and plated. The prepared slips were imaged. Alignment of neural cells on the patterned surface was observed via confocal microscopy (see FIG. 3).

### Example 1

### Master Preparation

Masters used to cast stamps were prepared by methods known in the art. (Moread, W. M., Semiconductor Lithography: Principles and Materials, Plenum, New York, 1988; Brambley et al., Adv. Mater. Opt. Electron., 4:55 (1994); Handbook of Microlithography, micromachining, and Microfabrication, Vol. 1 (Ed: P. Rai-Choudbury), SPEI Optical Engineering Press, Bellingham, Wash. (1997)). Briefly, a master may be fabricated on polished silicon wafers using AS P4620 photoresist (Clariant, Inc.) which are spin coated to a thickness of about 5 mm and processed by contact photolithography.

### Example 2

### Polymer Preparation

PMMA (M_{w} 120,000) was obtained from Aldrich Chemical Co. in pellet form and used as received. PMMA (100 mg) was compressed into thin films between highly polished steel plates at 10,000 pounds for 95 seconds using a laboratory press (Carver, Wabash, Ind.) heated to 150° C. The press was then cooled to 90° C., after which the plates were immediately removed and further cooled. The polymer film, with a thickness of 150 to 200 mm, was cut into 2-cm squares.

### Example 3

### Polydimethylsiloxane (PDMS) Stamp Preparation

The master is placed in a petri dish. In a separate container, PDMS monomer (Sylgard 184, Dow Coming, Midland, Mich.) is mixed with the curing agent provided with the PDMS monomer at a 10:1 ratio by weight. Bubbles arising from the mixing process are removed in a vacuum oven (Sheldon MFG, Aloha, Oreg.) at 28″ Hg and at room temperature. This mixture is then poured over the master; any arising bubbles are removed via vacuum at 28″ Hg; and the mixture and master are baked in an oven at 60° C. for a minimum of 4 hours. The resulting PDMS stamp is released from the master by cutting the PDMS with a sharp blade and peeling it from the master.

### Example 4

### Stamping Procedure

The PDMS stamp is placed along with PMMA in a low temperature plasma cleaner (Plasma Therm Plasma Processing Reactor, International Plasma Corp., Kresson, N.J.). To temporarily increase polarity, the PDMS stamp and PMMA are exposed to oxygen plasma (4 cc/minute) for 30 seconds at 200 W and room temperature. Laminin solution (50 mg/ml, Collaborative Biomedical, Bedford, Mass.) is pipetted directly onto the PDMS stamp or the stamp is dipped in the solution. Any excess solution is then removed via a stream of gas such as nitrogen. The PDMS stamp is then placed in contact with the PMMA and left for approximately 15 minutes to transfer the laminin onto the PMMA. The stamp is then removed. The laminin patterned PMMA can be used immediately or stored in Hank's balanced salt solution (HBSS, Gibco).

### Example 5

### Cell Culture

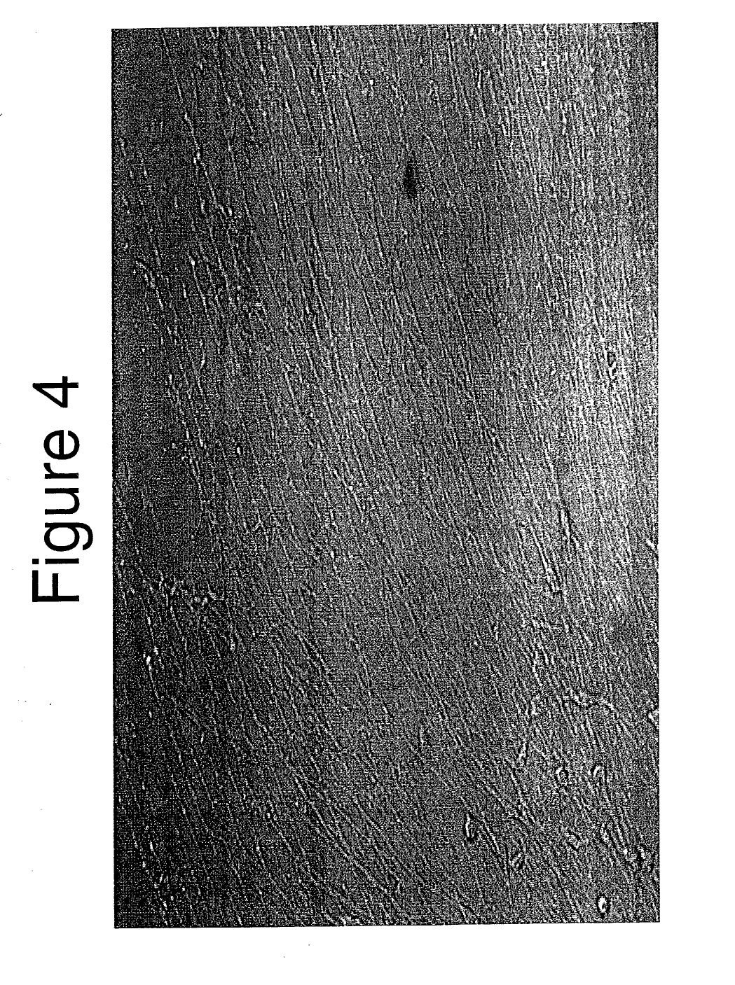

The laminin stamped PMMA squares were rinsed with Hanks's balanced saline solution and plated with dorsal root ganglia or dissociated neurons in minimal, serum-free media. The samples were incubated at 37° C. for 24 to 48 hours, imaged with a Zeiss laser scanning confocal microscope, and the images analyzed for pattern adherence. Cells had adhered to the laminin pattern on the PMMA substrate (FIG. 3). Moreover, neuronal processes also adhered and grew along the laminin pattern on the PMMA substrate (FIG. 4).

### Example 6

### Fluorescence Labeling

PMMA surfaces with cultured dorsal root ganglia were rinsed with phosphate buffered saline (PBS) and fixed with 4% formaldehyde. The primary antibody was pipetted onto the surface and incubated at room temperature in darkness for 1 hour. Surfaces were then rinsed three times at 15 minutes each with PBS and a secondary fluorescence-conjugated goat antibody was pipetted onto the surface and incubated at room temperature in darkness for 1 hour. The surfaces were imaged on a Zeiss laser scanning confocal microscope for fluorescence at 512 nm. The observed fluorescence was associated with the cells adhered to the PMMA substrate (FIG. 2).

What is claimed is:

1. An article comprising a pre-selected pattern of biologically active molecules stably adsorbed on a polymeric substrate.

2. An article comprising a pre-selected pattern of biologically active molecules stably adsorbed on a polymeric substrate by nonspecific molecular interaction.

3. An article comprising a pre-selected pattern of biologically active molecules stably adsorbed on a polymeric substrate, wherein said article has a non-raised surface.

4. The article of any one of claims 1-3 wherein said pre-selected pattern comprises a bound polymeric substrate surface having biologically active molecules stably adsorbed thereto and an unbound polymeric substrate surface having substantially no biologically active molecules adsorbed thereto.

5. The article of claim 4 wherein the surface of said unbound polymeric substrate is hydrophobic before and after said biologically active molecules have been stably adsorbed to said bound polymeric substrate surface.

6. The article of any one of claims 1-3 wherein said pattern is micron-sized.

7. The article of any one of claims 1-3 wherein said polymeric substrate is not polydimethylsiloxane.

8. The article of any one of claims 1-3 wherein the polymeric substrate is polyacrylate, polymethylacrylate, polycarbonate, polystyrene, polyhydroxy acid, polyanhydride, polyorthoester, polyphosphazene, polyphosphate, polyester, or a mixture thereof.

9. The article of any one of claims 1-3 wherein the polymeric substrate is biodegradable.

10. The article of any one of claims 1-3 wherein said biologically active molecules are hormones, extracellular matrix molecules, cell adhesion molecules, natural polymers, enzymes, peptides, antibodies, antigens, polynucleotides, growth factors, synthetic polymers, polylysine, drugs, or combinations thereof.

11. The article of any one of claims 1-3 wherein said biologically active molecules inhibit cell adhesion, growth or differentiation.

12. The article of any one of claims 1-3 wherein the pattern comprises 2-10 different types of biologically active molecules.

13. The article of any one of claims 1-3 wherein said pattern of biologically active molecules is a line.

14. The article of claim 14 wherein the line is from 5 micron to about 50 microns in length.

15. The article of claim 14 wherein the line is from 1 to 50 nm in width.

16. The article of any one of claims 1-3 wherein said pattern comprises a line, circle, oval, square, rectangle, diamond, triangle.

17. The article of any one of claims 1-3 wherein said polymeric substrate is a disc, sphere, sheet, tube, trough, dish.

18. The article of any one of claims 1-3 wherein said biologically active molecules comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

19. An apparatus comprising a stamp coated with biologically active molecules and an article comprising a pre-selected pattern of biologically active molecules stably adsorbed on a polymeric substrate; wherein said stamp has said pre-selected pattern.

20. The apparatus of claim 18 wherein the pre-selected pattern on said stamp is a raised surface.

21. The article of claims 18 wherein said biologically active molecules comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

22. A method for preparing a pattern of biologically active molecules on the surface of a polymeric substrate, which comprises exposing the surface of a polymeric substrate to conditions that increase the polarity of said surface, and contacting said surface with a pre-selected pattern of biologically active molecules.

23. A method for preparing a pattern of biologically active molecules on a hydrophobic polymeric substrate which comprises:
a) exposing a stamp and a hydrophobic polymeric substrate to conditions that increase the polarity, to a polar surface on said polymeric substrate;
b) coating a stamp with biologically active molecules to generate a coated stamp;
c) contacting said coated stamp with said polar surface under conditions sufficient to transfer said biologically active molecules to said polar surface; and
d) thereby creating a pattern of biologically active molecules on a hydrophobic polymeric substrate.

24. The method of claim 22 or 23 wherein said conditions that increase the polarity comprise exposure to a gaseous plasma.

25. The method of claim 22 or 23 wherein said conditions that increase the polarity comprise exposure to argon, nitrogen, or oxygen plasma.

26. The method of claim 22 or 23 wherein said conditions that increase the polarity further comprise exposure to 1-10 cc/minute oxygen plasma for 5-120 seconds at 50-400 W and at room temperature.

27. The method of claims 22 or 23 wherein said pattern comprises a bound polymeric substrate surface having biologically active molecules stably adsorbed thereto and an unbound polymeric substrate surface having substantially no biologically active molecules adsorbed thereto.

28. The method of claim 23 wherein the surface of said unbound polymeric substrate is hydrophobic before and after said biologically active molecules have been stably adsorbed to said bound polymeric substrate surface.

29. The method of claims 22 or 23 wherein said pattern is micron-sized.

30. The method of claims 22 or 23 wherein said polymeric substrate is not polydimethylsiloxane.

31. The method of claims 22 or 23 wherein the polymeric substrate is polyacrylate, polymethylacrylate, polycarbonate, polystyrene, polyhydroxy acid, polyanhydride, polyorthoester, polyphosphazene, polyphosphate, polyester, or a mixture thereof.

32. The method of claims 22 or 23 wherein the polymeric substrate is biodegradable.

33. The method of claims 22 or 23 wherein said biologically active molecules are hormones, extracellular matrix molecules, cell adhesion molecules, natural polymers, enzymes, peptides, antibodies, antigens, polynucleotides, growth factors, synthetic polymers, polylysine, drugs, or combinations thereof.

34. The method of claims 22 or 23 wherein said biologically active molecules inhibit cell adhesion, growth or differentiation.

35. The method of claims 22 or 23 wherein said biologically active molecules comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

36. The method of claims 22 or 23 wherein the pattern comprises 2-10 different types of biologically active molecules.

37. The method of claims 22 or 23 wherein said pattern of biologically active molecules is a line.

38. The method of claim 33 wherein the line is from 5 micron to 50 microns in length.

39. The method of claim 33 wherein the line is from 1 to 50 nm in width.

40. The method of claims 22 or 23 wherein said pattern comprises a line, circle, oval, square, rectangle, diamond, triangle.

41. The method of claims 22 or 23 wherein said polymeric substrate is a disc, sphere, sheet, tube, trough, dish.

42. An article made by the method of claim 22 or 23.

43. A method to spatially modulate the growth of a cell which comprises contacting a cell with the article of claim 1, 2 or 3 for a time and under conditions sufficient to adhere said cell to said biologically active molecules and to grow said cell along the micron-sized pattern of biologically active molecules on said polymeric substrate.

44. The method of claim 39 which further comprises implanting said article a mammal.

45. The method of claim 39 which further comprises implanting said article in a human.

46. The method of claim 39 wherein said cell is a nerve cell, an epithelial cell, a mesenchymal stem cell or a fibroblast cell.

47. The method of claim 39 wherein said cell is a nerve cell.

48. The method of claims 39 wherein said biologically active molecules comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

49. A method to regenerate a tissue which comprises contacting cells of said tissue with the article of claim 1, 2 or 3 for a time and under conditions sufficient to adhere said cells to biologically active molecules stably adsorbed to a polymeric substrate surface of said article and to grow said cells in a pre-selected pattern of biologically active molecules on said polymeric substrate.

50. The method of claim 45 wherein said pre-selected pattern comprises a shape missing from said tissue.

51. The method of claim 45 wherein said pre-selected pattern comprises a normal shape for said tissue.

52. The method of claim 45 which comprises implanting said article a mammal.

53. The method of claim 45 which comprises implanting said article in a human.

54. The method of claim 45 wherein said cells are nerve cells, epithelial cells, mesenchymal stem cells, or fibroblast cells.

55. The method of claim 45 wherein said cell is a nerve cell.

56. The method of claim 45 wherein said biologically active molecules comprise SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.


SEQUENCE LISTING (13 sequences)

SEQ ID NO: 1 - protein, 5 aa, Unknown
  A peptide sequence found in the B1 chain of laminin.
YIGSR

SEQ ID NO: 2 - protein, 5 aa, Unknown
  A peptide sequence found in the A chain of laminin.
IKVAV

SEQ ID NO: 3 - protein, 19 aa, Unknown
  A peptide sequence found in the A chain of laminin.
CSRARKQAASIKVAVSADR

SEQ ID NO: 4 - protein, 4 aa, Unknown
  A peptide sequence found in fibronectin.
REDV

SEQ ID NO: 5 - protein, 4 aa, Unknown
  A peptide sequence found in fibronectin.
RGDS

SEQ ID NO: 6 - protein, 4 aa, Unknown
  A peptide sequence found in fibronectin.
REDV

SEQ ID NO: 7 - protein, 4 aa, Unknown
  A peptide sequence found in vitronectin.
RGDV

SEQ ID NO: 8 - protein, 5 aa, Unknown
  A peptide sequence found in laminin A.
LRGDN

SEQ ID NO: 9 - protein, 5 aa, Unknown
  A peptide sequence found in laminin B1.
PDSGR

SEQ ID NO: 10 - protein, 4 aa, Unknown
  A peptide sequence found in collagen 1.
RGDT

SEQ ID NO: 11 - protein, 4 aa, Unknown
  A peptide that can mediate adhesion of platelets and other cells.
DGEA

SEQ ID NO: 12 - protein, 4 aa, Unknown
  A peptide sequence found in thrombospondin.
VTXG

SEQ ID NO: 13 - protein, 8 aa, Unknown
  A peptide sequence found in laminin B2.
RNIAEIIK